SEQUENCE LISTING (2 sequences)

SEQ ID NO: 1 - protein, 366 aa, Bacillus sphaericus
MTLEIFEYLEKYDYEQVVFCQDKESGLKAIIAIHDTTLGPALGGTRMWTYDSEEAAIEDALRLAKGMTYKNAAAGLNLGGAKTVIIGDPRKDKSEAMFRALGRYIQGLNGRYITAEDVGTTVDDMDIIHEETDFVTGISPSFGSSGNPSPVTAYGVYRGMKAAAKEAFGTDNLEGKVIAVQGVGNVAYHLCKHLHAEGAKLIVTDINKEAVQRAVEEFGASAVEPNEIYGVECDIYAPCALGATVNDETIPQLKAKVIAGSANNQLKEDRHGDIIHEMGIVYAPDYVINAGGVINVADELYGYNRERALKRVESIYDTIAKVIEISKRDGIATYVAADRLAEERIASLKNSRSTYLRNGHDIISRR

SEQ ID NO: 2 - DNA, 1723 bp, Bacillus sphaericus
cccgggcaataggaatcgacttgccaaaagtggcaccaattgcagcagtagaagttgtgaatccagcgatgcaggcgacaattgatgcagcgatgttaacacaaatgaatcgccgcggacaaattaaagattgtatcgttgatggaccacttgctttagataatgcagtatcaacaaattgcagcagagcatcaaaggcatagtaagtgatgtagcaggtaaggcagatattttactcgtcccaacgattgaagctggaaatgtgctatataaatcactcgtatactttgcggatgcaaaagtaggagcaatgattgctggcgcaaaagcaccgattgttttaacatctcgtgctgattcagcagaaacaaaagtatattcattagcattggcagttgcgactgcttcaaaataaaccaaaaagggtaaaacattagggggaaacgacaatgacattagaaatcttcgaatacttagaaaaatatgattatgagcaagtagtattttgtcaagataaagaatctggtttaaaagcaattattgcaattcatgatacaacacttggaccggctcttggtggaacaagaatgtggacatatgattctgaagaagcggcgattgaagatgcattgcgtcttgcaaaagggatgacatacaaaaacgcagcagctggtttaaacttaggtggtgcgaaaacagtaattatcggtgatcctcgtaaagataagagcgaagcaatgttccgtgcactaggacgttatatccaaggactaaacggacgttacattacagctgaagatgttggtacaacagtagatgatatggatattatccatgaagaaactgactttgtaacaggtatctcaccatcattcggttcttctggtaacccatctccggtaactgcatacggtgtttaccgtggtatgaaagcagctgcaaaagaagctttcggtactgacaatttagaaggaaaagtaattgctgttcaaggcgttggtaacgtagcatatcacctatgcaaacatttacacgctgaaggagcaaaattaattgttacagatattaataaagaagctgtacaacgtgctgtagaagaattcggtgcatcagcagttgaaccaaatgaaatttacggtgttgaatgcgatatttacgcaccatgtgcactaggcgcaacagttaatgatgaaactattccacaacttaaagcaaaagtaatcgcaggttctgcgaataaccaattaaaagaagatcgtcatggtgacatcattcatgaaatgggtattgtatacgcaccagattatgtaattaatgcaggtggcgtaattaacgtagcagacgaattatatggatacaatagagaacgtgcactaaaacgtgttgagtctatttatgacacgattgcaaaagtaatcgaaatttcaaaacgcgatggcatagcaacttatgtagcggcagatcgtctagctgaagagcgcattgcaagcttgaagaattctcgtagcacttacttacgcaacggtcacgatattattagccgtcgctaatcattcatatataacttttacaaaaggtgtggttacctcttatgaggtttccacttccttttgaatttattagtggaggtagcaacattgtctgtaaatcgaattcttgttattaacccaggtagtacatccacaaaaattggtgtttttgataatgaaagacccgttctaga

(19) United States
(12) Patent Application Publication
(10) Pub. No.: US 2016/0319312 A1
(43) Pub. Date: Nov. 3, 2016

(54) SYNTHESIS METHOD FOR L-CYCLIC ALKYL AMINO ACID AND PHARMACEUTICAL COMPOSITION HAVING THEREOF

(71) Applicants: ASYMCHEM LABORATORIES (TIANJIN) CO., LTD, Teda, Tianjin (CN); ASYMCHEM LIFE SCIENCE (TIANJIN) CO., LTD, Teda, Tianjin (CN); TIANJIN ASYMCHEM PHARMACEUTICAL CO., LTD, Tianjin (CN); ASYMCHEM LABORATORIES (FUXIN) CO., LTD, Fuxin, Liaoning (CN); JILIN ASYMCHEM LABORATORIES CO., LTD., Dunhua, Jilin (CN)

(72) Inventors: Hao Hong, Teda, Tianjin (CN); Changsheng Zheng, Teda, Tianjin (CN); Lina Guo, Teda, Tianjin (CN)

(21) Appl. No.: 14/783,653

(22) PCT Filed: Jul. 29, 2013
(86) PCT No.: PCT/CN2013/080358

§ 371 Date: Oct. 9, 2015

(51) Int. Cl. C12P 13/00; A61K 31/351; A61K 31/198

(52) U.S. Cl. CPC C12P 13/005; A61K 31/198; A61K 31/351

(57) ABSTRACT

A synthesis method for L-cyclic alkyl amino acid and a pharmaceutical composition having the said amino acid are provide in the present disclosure provides. The synthesis method comprises: step A.) preparing a cyclic alkyl keto acid or a cyclic alkyl keto acid salt having Structural Formula (I) or Structural Formula (II), and step B.) mixing the cyclic alkyl keto acid or the cyclic alkyl keto acid salt with ammonium formate, a leucine dehydrogenase, a formate dehydrogenase and a coenzyme NAD⁺, and carrying out a reductive amination reaction to generate the L-cyclic alkyl amino acid, wherein the Structural Formula (I) is

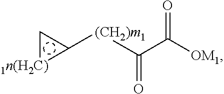

where n₁≧1, m₁≧0 and the M₁ is H or a monovalent cation; the Structural Formula (II) is

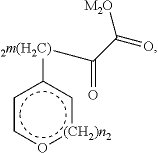

where n₂≧0, m₂≧0, the M₂ is H or a monovalent cation, an amino acid sequence of the leucine dehydrogenase is SEQ ID No.1.

This application is a U.S. National Phase application from International PCT Patent Application No. PCT/CN2013/080358 filed on Jul. 29, 2013. The entire disclosure of the above application, including the sequence listing, is incorporated herein by reference. A new computer readable form of the sequence listing is not filed herewith as the sequence listing is identical to a compliant computer readable sequence listing filed with PCT Patent Application No. PCT/CN2013/080358. Use of the compliant computer readable sequence listing on file is hereby requested. A paper copy is filed herewith.

### FIELD

The present disclosure relates to the field of medicine synthesis, and particularly to a synthesis method for L-cyclic alkyl amino acid and a pharmaceutical composition having the said amino acid.

### BACKGROUND

At present, non-natural chiral cyclic alkyl amino acids are mainly synthesized chemically, including methods such as single-configuration conversation implemented on a certain key intermediate by asymmetric catalytic hydrogenation of a noble metal, resolution of a racemate by using a chiral reagent, asymmetric synthesis using a chiral auxiliary, rational synthesis using a chiral raw material, and the like. However, these methods have the following disadvantages:

(1) implementation of single-configuration conversation for a certain key intermediate by an asymmetric catalytic hydrogenation of a noble metal has the following disadvantages: the noble metal asymmetric catalyst is expensive, a large amount of organic solvent is needed in the reaction, there are heavy metal residues in a product and there may be excessive reduction by-products in the product; in addition, binding of the noble metal and a ligand is usually interfered by a heterocycle contained in a synthesis raw material, which results in low catalytic efficiency;

(2) an isomer required in a racemate is obtained by applying a traditional chiral resolving method, which may cause waste of the other half of raw materials;

(3) asymmetric synthesis using a chiral auxiliary or a chiral raw material involves expensive chiral raw materials, long synthesis route and a large amount of organic solvent, in addition, products obtained in synthesis of some cyclic alkyl amino acids are low in optical purity, or the products can be hardly separated from impurities.

It is also reported in some literatures in the prior art that some simple alkyl keto acids are catalyzed by specific enzymes to be converted into corresponding amino acids through biosynthesis. However, in the prior art, because the cyclic alkyl amino acids have relatively special properties, there are no proper enzymes and corresponding reaction conditions can be used in biotransformation for synthesizing chiral cyclic alkyl amino acid.

### SUMMARY

The present disclosure aims at providing a synthesis method for L-cyclic alkyl amino acid and a pharmaceutical composition having the said amino acid to obtain the L-cyclic alkyl amino acid with relatively high optical purity.

To realize the purpose above, a synthesis method for the L-cyclic alkyl amino acid is provided according to an aspect of the present disclosure. The synthesis method comprises: Step A: preparing a cyclic alkyl keto acid or a cyclic alkyl keto acid salt having Structural Formula (I) or Structural Formula (II); Step B: mixing the cyclic alkyl keto acid or the cyclic alkyl keto acid salt with ammonium formate, a leucine dehydrogenase, a formate dehydrogenase and a coenzyme Nicotinamide Adenine Dinucleotide (NAD⁺), and carrying out a reductive amination reaction to generate the L-cyclic alkyl amino acid, wherein the Structural Formula (I) is

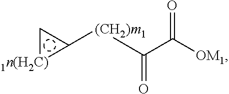

where n₁≧1, m₁≧0 and the M₁ is H or a monovalent cation;the Structural Formula (II) is

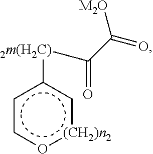

where n₂≧0, m₂≧0, the M₂ is H or a monovalent cation, and an amino acid sequence of the leucine dehydrogenase is SEQ ID No. 1.

Further, a gene sequence coding the leucine dehydrogenase is SEQ ID No. 2.

Further, an expression process of the leucine dehydrogenase comprises: inserting a Deoxyribonucleic Acid (DNA) fragment containing the gene sequence SEQ ID No. 2 into a vector to obtain a gene recombinant plasmid; transferring the gene recombinant plasmid to a host strain and culturing the host strain on a culture medium, and inducing production of the leucine dehydrogenase by an inducer; breaking the host strain with ultrasonic waves, and then carrying out centrifugal separation to obtain a crude enzyme mixed solution which contains the leucine dehydrogenase and the formate dehydrogenase.

Further, in the crude enzyme mixed solution, the specific enzyme activity of the leucine dehydrogenase is 50 U/ml to 100 U/ml and the specific enzyme activity of the formate dehydrogenase is 20 U/ml to 50 U/ml.

Further, the Step B comprises: adding the cyclic alkyl keto acid or the cyclic alkyl keto acid salt and ammonium formate to water, mixing and then regulating the pH value to 8.0 to 8.5 to obtain a mixed solution, adding the crude enzyme mixed solution and the coenzyme NAD⁺ to the mixed solution and performing reaction at 30 to 40° C. to generate the L-cyclic alkyl amino acid.

Further, 2 ml to 12 ml of the crude enzyme mixed solution is added to each mole of the cyclic alkyl keto acid; 0.005 mole to 0.1 mole of the coenzyme NAD⁺ is added to each mole of the cyclic alkyl keto acid and 1.5 moles to 5 moles of ammonium formate is added to each mole of the cyclic alkyl keto acid.

Further, after the Step B, the synthesis method further comprises: adding concentrated hydrochloric acid to a system after the reaction to regulate the pH value of the system to be smaller than or equal to 1, passing the system with the pH value of smaller than or equal to 1 through diatomite to obtain a filtrate; regulating the pH value of the filtrate to 5.0 to 7.0, then passing the filtrate with the pH value of 5.0 to 7.0 through a strong acid cation exchange resin to obtain a crude product; concentrating the crude product, adding an alcoholic solvent to wash the concentrated crude product and drying the washed crude product to obtain a purified L-cyclic alkyl amino acid.

Further, a method for preparing the cyclic alkyl keto acid having the Structural Formula (I) comprises the following steps: preparing a Grignard reagent of a cyclic alkyl halogen compound having Structural Formula (III); subjecting the Grignard reagent and diethyl oxalate to a substitution reaction to obtain an intermediate product; subjecting the intermediate product to a hydrolysis reaction to obtain the cyclic alkyl keto acid, wherein the Structural Formula (III) is

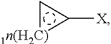

where n₁≧1 and the X is halogen.

Further, the n₁ in the Structural Formula (III) is 1 or 2, and the intermediate product is subjected to the hydrolysis reaction under the action of a biological enzyme.

Further, the n1 in the Structural Formula (III) is larger than or equal to 3, and the intermediate product is subjected to the hydrolysis reaction under the action of sodium hydroxide or potassium hydroxide.

Further, a method for preparing the cyclic alkyl keto acid salt having the Structural Formula (I) comprises: preparing a Grignard reagent of the cyclic alkyl halogen compound having Structural Formula (III); subjecting the Grignard reagent and diethyl oxalate to a substitution reaction to obtain an intermediate product; subjecting the intermediate product to a hydrolysis reaction to obtain the cyclic alkyl keto acid; enabling the cyclic alkyl keto acid to react with CH₃OM₁′ or M₁′OH to obtain the cyclic alkyl keto acid salt, wherein the Structural Formula (III) is

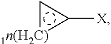

where n₁≧1, the X is a halogen and the M₁′ is a monovalent cation.

Further, in the Structural Formula (III), 3≧n₁≧5, and the intermediate product is subjected to the hydrolysis reaction under the action of sodium hydroxide or potassium hydroxide.

Further, the cyclic alkyl keto acid salt is a cyclopropyl keto acid salt and a method for preparing the cyclopropyl keto acid salt comprises the following step: oxidizing cyclopropyl methyl ketone in an alkaline condition to obtain the cyclopropyl keto acid salt.

Further, KMnO₄ is applied as an oxidant in the oxidation process and the oxidant is reduced until it disappears after the oxidation reaction.

Further, the method for preparing the cyclic alkyl keto acid salt having the Structural Formula (I) comprises the following steps: subjecting cyclic alkyl formaldehyde to a reduction reaction to obtain cyclic alkyl methyl alcohol; subjecting the cyclic alkyl methyl alcohol to a halogenation reaction to obtain a second cyclic alkyl methyl halogen compound having Structural Formula (IV); preparing a Grignard reagent of the second cyclic alkyl methyl halogen compound; subjecting the Grignard reagent and diethyl oxalate to a substitution reaction to obtain an intermediate product; subjecting the intermediate product to a hydrolysis reaction to obtain the cyclic alkyl keto acid; enabling the cyclic alkyl keto acid to react with CH₃OM₁′ or M₁′OH to obtain the cyclic alkyl keto acid salt, wherein the Structural Formula (IV) is

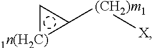

where n₁≧1, m₁=1, the X is a halogen, and the M₁′ is a monovalent cation.

Further, the method for preparing the cyclic alkyl keto acid salt having the Structural Formula (I) further comprises a process for preparing the cyclic alkyl formaldehyde,the process comprises: preparing a Grignard reagent of a cyclic alkyl halogen compound having Structural Formula (III), enabling the Grignard reagent to have a Bouveault aldehyde synthesis reaction to obtain the cyclic alkyl formaldehyde, wherein the Structural Formula (III) is

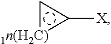

where n₁≧1, the X is a halogen.

Further, a method for preparing the cyclic alkyl keto acid having the Structural Formula (II) comprises the following steps: subjecting carboxylic acid having Structural Formula (V) to a Grignard reagent addition reaction to obtain an intermediate product; subjecting the intermediate product to a hydrolysis reaction in an alkaline condition to obtain the cyclic alkyl keto acid, wherein the Structural Formula (V) is

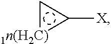

where n₂≧0 and m₂≧0.

Further, n₂=1 and m₂=0.

A pharmaceutical composition is provided according to another aspect of the present disclosure, the pharmaceutical composition comprises an effective dose of an L-cyclic alkyl amino acid and a pharmaceutical vector, the L-cyclic alkyl amino acid is prepared according to the synthesis method above.

Applying the technical solution of the present disclosure, a specific leucine dehydrogenase having the amino acid sequence of SEQ ID No. 1, the formate dehydrogenase and the coenzyme NAD⁺ are used together to enable a reductive amination reaction of a cyclic alkyl keto acid so as to generate an L-cyclic alkyl amino acid. A chiral center is formed through conversion catalyzed by the leucine dehydrogenase and the coenzyme. The conversion rate of raw materials is as high as above 80% with high chiral selectivity; In addition, it only needs two steps to synthesize the L-cyclic alkyl amino acid by using a keto acid as a substrate. At the same time, the conversion rate of the raw materials is high, thus it does not need to separate and purify an isomer from an obtained product, thus further simplifying a synthesis process of the L-cyclic alkyl amino acid. In addition, reaction conditions in the whole synthesis process are moderate, which is more applicable to mass industrial production of the L-cyclic alkyl amino acid.

### DETAILED DESCRIPTION OF THE EMBODIMENTS

It should be noted that, if there is no conflict, the embodiments of the application and the characteristics in the embodiments can be combined with each other. The present disclosure will be described in details below in combination with the embodiments. Here, it should be explained that, similar to a conventional representing method in the art, a dashed line ring in a ring-shaped structure may be connected with any one of the carbon positions in the ring-shaped structure.

In a typical embodiment of the present disclosure, a synthesis method for L-cyclic alkyl amino acid is provided. The synthesis method comprises: Step A: preparinga cyclic alkyl keto acid or a cyclic alkyl keto acid salt having Structural Formula (I) or Structural Formula (II); Step B: mixing the cyclic alkyl keto acid or the cyclic alkyl keto acid salt with ammonium formate, a leucine dehydrogenase, a formate dehydrogenase and a coenzyme NAD⁺, and carrying out a reductive amination reaction to generate the L-cyclic alkyl amino acid,

wherein the Structural Formula (I) is

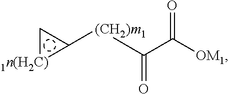

where n₁≧1, m₁≧0 and the M₁ is H or a monovalent cation; the Structural Formula (II) is

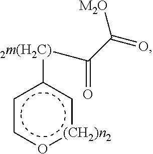

where n₂≧0, m₂≧0, the M₂ is H or a monovalent cation, and an amino acid sequence of the leucine dehydrogenase is SEQ ID No. 1.

The synthesis method subjectes specific leucine dehydrogenase having the amino acid sequence of SEQ ID No. 1, the formate dehydrogenase and the coenzyme NAD⁺ together to reductive amination reaction of the cyclic alkyl keto acid so as to generate the L-cyclic alkyl amino acid. A chiral center is formed through conversion catalyzed by the leucine dehydrogenase and the coenzyme. The conversion rate of raw materials is as high as above 80% with high chiral selectivity. In addition, it only needs two steps to synthesize the L-cyclic alkyl amino acid by using a keto acid as a substrate. At the same time, the conversion rate of the raw materials is high, thus it does not need to separate and purify an isomer from an obtained product, thus further simplifying a synthesis process of the L-cyclic alkyl amino acid. In addition, reaction conditions in the whole synthesis process are moderate, which is more applicable to mass industrial production of the L-cyclic alkyl amino acid. The monovalent cation which may be applied to the present application comprises, but is not limited to an alkali metal ion, an amino and a mercapto etc., and a monovalent cation which can form a salt with an acid can be applied in the present disclosure.

In the synthesis method, a gene sequence coding the leucine dehydrogenase is SEQ ID No. 2. The leucine dehydrogenase coded by the gene sequence has higher selectivity and catalytic conversion rate for catalyzing the synthesis of the L-cyclic alkyl amino acid from the cyclic alkyl keto acid and ammonium formate.

In a preferred embodiment of the present disclosure, an expression process of the leucine dehydrogenase comprises: inserting a DNA fragment containing the gene sequence SEQ ID No. 2 into a vector to obtain a gene recombinant plasmid; transfering the gene recombinant plasmid to a host strain and culturing the host strain on a culture medium, and inducing production of the leucine dehydrogenase by an inducer; breaking the host strain with ultrasonic waves, and then carrying out centrifugal separation to obtain a crude enzyme mixed solution which contains the leucine dehydrogenase and the formate dehydrogenase.

The gene recombinant plasmid obtained by inserting the DNA fragment containing the gene sequence into the vector to obtain the gene recombinant plasmid, the leucine dehydrogenase obtained from the gene recombinant plasmid matched with the induction with the inducer, and the activity and content of the leucine dehydrogenase are relatively high. The crude enzyme mixed solution obtained after breaking the host strain and performing the centrifugation not only contains the leucine dehydrogenase, but also contains the formate dehydrogenase contained in the nutritional methyl host strain itself. The present disclosure can catalyze conversion of a keto acid into an amino acid by using the crude enzyme mixed solution directly.

During an implementation process of the embodiment, the specific enzyme activities of both the leucine dehydrogenase and the formate dehydrogenase in the crude enzyme solution may be influenced by changes of the temperature and the culture medium, all crude enzyme mixed solutions obtained may be applied to the present disclosure, and in a preferred crude enzyme mixed solution obtained, the enzyme specific activity of the leucine dehydrogenase is 50 U/ml to 100 U/ml and that of the formate dehydrogenase is 20 U/ml to 50 U/ml. The crude enzyme mixed solution having the enzyme specific activities has higher chiral selectivity and catalytic efficiency in a conversion process of the keto acid into the L-cyclic alkyl amino acid.

In another preferred embodiment of the present disclosure, the Step B in the synthesis method comprises: adding the cyclic alkyl keto acid or the cyclic alkyl keto acid salt and ammonium formate to water, regulating the pH value to 8.0 to 8.5, adding the crude enzyme mixed solution and the coenzyme NAD⁺ and processing reaction at 30 to 40° Cuntil conversion of the raw materials is finished to generate the L-cyclic alkyl amino acid. In the Step B, the water is used as a solvent, thus greatly reducing production costs and avoiding production of an organic solvent. The synthesis process is green environment protection, which is further applicable to mass industrial production.

In order to further control costs of raw materials and regulate and control the proportion of each raw material to produce a product as much as possible, preferably, 2 ml to 12 ml of the crude enzyme mixed solution is added to each gram of the cyclic alkyl keto acid; 0.005 mole to 0.1 mole of the coenzyme NAD⁺ is added to each mole of the cyclic alkyl keto acid and 1.5 moles to 5 moles of ammonium formate is added to each mole of the cyclic alkyl keto acid.

In another preferred embodiment of the present disclosure, after the Step B, the synthesis method further comprises: adding concentrated hydrochloric acid to a system after the reaction to regulate the pH value of the system to be smaller than or equal to 1, pass the system with the pH value of smaller than or equal to 1 through diatomite to obtain a filtrate; regulating the pH value of the filtrate to 5.0 to 7.0, then pass the filtrate with the pH value of 5.0 to 7.0 through a strong acid cation exchange resin to obtain a crude product;concentrating the crude product, regulating the pH value to 7.0, adding an alcoholic solvent to wash the crude product and drying the wased crude product to obtain a purified L-cyclic alkyl amino acid. Since the present disclosure has higher chiral selectivity, and does not need to separate and purify the isomer during separation and purification of the product, thus the separation method of the present disclosure is simple, and it only needs to separate the product from the enzymes and raw materials etc.

In another preferred embodiment of the present disclosure, a method for preparing the cyclic alkyl keto acid having the Structural Formula (I) in the synthesis method comprises the following steps: preparing a Grignard reagent of a cyclic alkyl halogen compound having Structural Formula (III); subjecting the Grignard reagent and diethyl oxalate to a substitution reaction to obtain an intermediate product; subjecting the intermediate product to a hydrolysis reaction to obtain the cyclic alkyl keto acid, wherein the Structural Formula (III) is

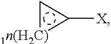

where n₁≧1 and the X is a halogen.

The synthesis route of the cyclic alkyl keto acid is relatively short without a noble metal catalyst, thereby ensuring that there are no heavy metal residues in the obtained cyclic alkyl keto acid; the use of a noble metal catalyst is also avoided in the subsequent synthesis process of the L-cyclic alkyl amino acid, thereby further ensuring that there are also no heavy metal residues in the obtained L-cyclic alkyl amino acid.

When cyclopropyl amino acid or cyclobutyl amino acid is prepared by using the production method above, the intermediate is preferably subjected to a hydrolysis reaction under the action of a biological enzyme. The reaction performed in the presence of the biological enzyme is not only more moderate in reaction conditions, but also easier to control the reaction rate and the yield of a reaction product. For example, cyclobutyl keto acid is synthesized by cyclobutyl oxoacetate catalyzed by Candida Antarctic.

When the production method uses the cyclic alkyl halogen compound having the Structural Formula (III) as a substrate to synthesize the cyclic alkyl keto acid, wherein n₁≧3, the intermediate is preferably subjected to the hydrolysis reaction under the action of a sodium hydroxide or a potassium hydroxide. The conversion efficiency of the intermediate product is relatively high when the hydrolysis reaction is performed under the action of an inorganic base including sodium hydroxide and potassium hydroxide etc.

In another preferred embodiment of the present disclosure, when the cyclic alkyl amino acid cannot be synthesized by using the cyclic alkyl keto acid as a substrate, a method for preparing the cyclic alkyl keto acid salt having the Structural Formula (I) in the synthesis method preferably comprises: preparing a Grignard reagent of the cyclic alkyl halogen compound having Structural Formula (III); subjecting the Grignard reagent and diethyl oxalate to a substitution reaction to obtain an intermediate product; subjecting the intermediate product to a hydrolysis reaction to obtain a cyclic alkyl keto acid; enabling the cyclic alkyl keto acid to react with CH₃OM₁′ or M₁′OH to obtain the cyclic alkyl keto acid salt, wherein the Structural Formula (III) is

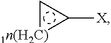

where n₁≧1, the X is a halogen and M₁′M₁ is a monovalent cation.

Similarly, the synthesis route of the cyclic alkyl keto acid is shorter than a synthesis route in the prior art and use of a noble metal catalyst is avoided, thereby ensuring that there are no heavy metal residues in the obtained cyclic alkyl keto acid; use of a noble metal catalyst is also avoided in the subsequent synthesis process of the L-cyclic alkyl keto acid, thereby further ensuring that there are also no heavy metal residues in the obtained L-cyclic alkyl keto acid.

When the method uses the cyclic alkyl halogen compound having the Structural Formula OM as a substrate to synthesize the cyclic alkyl keto acid, where 3≦n₁≦5, the intermediate is preferably subjected to the hydrolysis reaction under the action of a sodium hydroxide or a potassium hydroxide. The conversion efficiency of the intermediate product is relatively high when the hydrolysis reaction is performed under the action of an inorganic base including sodium hydroxide and potassium hydroxide etc.

In another preferred embodiment of the present disclosure, the cyclic alkyl keto acid salt is a cyclopropyl keto acid salt and a method for preparing the cyclopropyl keto acid salt comprises the following step: oxidizing cyclopropyl methyl ketone in an alkaline condition to obtain the cyclopropyl keto acid salt. The present embodiment uses cyclopropyl methyl ketone as a substrate to synthesize cyclopropyl amino acid, it only needs to subject cyclopropyl methyl ketone to the oxidation reaction in the alkaline condition to obtain the cyclopropyl keto acid salt, and then mix the cyclopropyl keto acid salt with ammonium formate, the leucine dehydrogenase, the formate dehydrogenase and the coenzyme NAD⁺ to enable the reductive amination reaction to generate the L-cyclic alkyl amino acid, the process only needs two steps without a heavy metal catalyst, thus further ensuring the chiral purity and yield of the L-cyclic alkyl amino acid.

KMnO4 is preferably applied as an oxidant in the process of oxidizing cyclopropyl methyl ketone in the alkaline condition to obtain the cyclopropyl keto acid salt, and the oxidant is reduced until it disappears after the oxidation reaction. In a practical operation, the skilled person in the art may use sodium hydroxide to create the alkaline condition and sodium bisulfite is preferably used as a reducer after the oxidation reaction to reduce unreacted KMnO₄. It only needs to filter the system after KMnO₄ is completely reduced, and a filtrate can be used in the reaction of the next step after being concentrated.

In another preferred embodiment of the present disclosure, the method for preparing the cyclic alkyl keto acid salt having the Structural Formula (I) comprises the following steps: subjecting cyclic alkyl formaldehyde to a reduction reaction to obtain cyclic alkyl methyl alcohol; subjecting the cyclic alkyl methyl alcohol to a halogenation reaction to obtain a second cyclic alkyl methyl halogen compound having Structural Formula (IV); preparing a Grignard reagent of the second cyclic alkyl methyl halogen compound; subjecting the Grignard reagent and diethyl oxalate to a substitution reaction to obtain an intermediate product; subjecting the intermediate product to a hydrolysis reaction to obtain a cyclic alkyl keto acid; enabling the cyclic alkyl keto acid to react with CH3OM₁′ or M₁′OH to obtain the cyclic alkyl keto acid salt, wherein the Structural Formula (IV) is

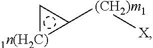

where n₁≧1, m₁=1, the X is a halogen, and the M₁′ is a monovalent cation.

The preparation method of the embodiment above uses a simple preparation method to obtain the cyclic alkyl keto acid salt without a heavy metal catalyst, and further, no noble metal catalyst is used in the process of using the cyclic alkyl keto acid salt to synthesize the cyclic alkyl amino acid, thereby ensuring that there are no heavy metal residues in the obtained cyclic alkyl keto acid; use of a noble metal catalyst is also avoided in the subsequent synthesis process of the L-cyclic alkyl keto acid, thereby further ensuring that there are also no heavy metal residues in the obtained L-cyclic alkyl keto acid.

The cyclic alkyl formaldehyde used in the embodiment may be synthesized by a method for synthesizing cyclic alkyl formaldehyde by using cycloolefine as a substrate in the prior art. Preferably, the method for preparing the cyclic alkyl keto acid salt having the Structural Formula (I) further comprises a process for preparing the cyclic alkyl formaldehyde. The preparing process comprises: preparing a Grignard reagent of the cyclic alkyl halogen compound having Structural Formula (III); enabling the Grignard reagent to have a Bouveault aldehyde synthesis reaction to obtain the cyclic alkyl formaldehyde, wherein the Structural Formula (III) is

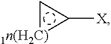

where n₁≧1, the X is a halogen. The preparation process has simple steps, easily-controlled operation conditions, and a high purity and a high yield of cyclic alkyl formaldehyde.

In a preferred embodiment of the present application, a method for preparing the cyclic alkyl keto acid having the Structural Formula (II) comprises the following steps: subjecting carboxylic acid having Structural Formula (V) to a Grignard reagent addition reaction to obtain an intermediate product; and subjecting the intermediate product to a hydrolysis reaction in an alkaline condition to obtain the cyclic alkyl keto acid, wherein the Structural Formula (V) is

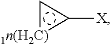

where n₂≧0 and m₂≧0. Using a heterocyclic alkyl group with a carboxyl group as a substrate to enable a Grignard reagent addition reaction and then to enable a hydrolysis reaction in an alkaline condition can obtain the cyclic alkyl keto acid with a relatively high conversation rate, thus further ensuring the purity of the obtained cyclic alkyl amino acid.

Preferably, the carboxylic acid having the Structural Formula (V) is 4-carboxypyran, i.e. n₂=1 and m₂=0, the 4-carboxypyran is dissolved in Tetrahydrofuran (THF) and subjected to a reaction with diisopropylamine under a catalytic action of I-PrMgCl/THF at a temperature between −10 and 0° C., ethyl oxalate is added to the system after the reaction to enable an addition reaction to obtain an intermediate product; the intermediate product is then subjected to a hydrolysis reaction in an alkaline condition formed by potassium hydroxide or sodium hydroxide to obtain 4-pyranyl keto acid.

A pharmaceutical composition is provided in another typical embodiment of the present disclosure, including an effective dose of an L-cyclic alkyl amino acid and a pharmaceutical vector, the L-cyclic alkyl amino acid is prepared according to the synthesis method above. The L-cyclic alkyl amino acid of the present disclosure is relatively high in purity, therefore the pharmaceutical composition having the L-cyclic alkyl amino acid has a smaller target and lower side effects compared with a pharmaceutical composition having an L-cyclic alkyl amino acid in the prior art.

The beneficial effect of the present disclosure will be further described hereinafter in combination with embodiments and comparison examples.

The leucine dehydrogenase used in the following embodiments is a leucine dehydrogenase having an amino acid sequence of SEQ ID No. 1, wherein a gene sequence coding the leucine dehydrogenase of the 1^{st} embodiment to the 7^{th} embodiment is from bacillus sphaericus.

An expression process of the leucine dehydrogenase is as follows:

DNA fragments containing the gene sequence of SEQ ID No. 2 were synthesized, and the synthesized DNA fragments were insert into pET-22b(+) vector to obtain gene recombinant plasmids; the gene recombinant plasmids were transferred to escherichia colis BL21, the escherichia colis BL21 were cultured on a culture medium, the leucine dehydrogenases were induced production by an inducer; the escherichia coils BL21 were broken with ultrasonic waves, and then centrifugal separation was carried out to obtain a crude enzyme mixed solution having the leucine dehydrogenase with a specific enzyme activity of 50 to 100 U/ml and a formate dehydrogenase with a specific enzyme activity of 20 to 50 U/ml.

### Embodiment 1

Synthesis of L-cyclopropyl Glycine

Step 1: 500 g of cyclopropyl methyl ketone, 15.6 g of NaOH and 8.7 L of water were added to a 20 L four-neck flask at room temperature to form a mixed system, the mixed system was heated to 45 to 55° C., and then 8.7 L of an aqueous solution having 1864.8 g of KMnO₄ was dropwise added to the mixed system, then undissolved KMnO₄ was added to the mixed system in batches, addition of KMnO₄ is finished in about 10 h, track the reaction until the raw materials fully react, the yield is 57.9% according to internal standard, then 10% of NaHSO₃ was dropwise added to the mixed system after the reaction to break unreacted KMnO₄; suction filtration was performed, a filtrate of 70% was concentrated and directly was use in the next step. IH MR (500 MHz, CD₃Cl):δ1.29 (m, 3H), 1.06(m, 1H), 0.95(m, 1H).

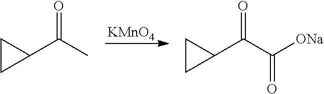

Step 2: 1627 g of a sodium cyclopropyloxo acetate aqueous solution having a concentration of 14.5%, which is obtained in the previous step, 193.3 g of ammonium formate, 2350 mL of a mixed crude enzyme solution having a leucine dehydrogenase with a specific enzyme activity of 60 U/ml and a formate dehydrogenase with a specific enzyme activity of 50 U/ml and 10.18 g of β-NAD⁺ were added to a 5 L four-neck flask at room temperature to form a reaction system, the pH value of the reaction system was regulated to 8.0 to 8.5 and heated to 30 to 40° C. the reaction system to reacted according to the following chemical formula, the system after reacting for 16 h was tracked and detected that reaction of the raw materials were finished, then cooled to room temperature, 750 mL of concentrated hydrochloric acid was slowly add to the mixed system after the reaction in a dropwise manner while stirring to reduce the pH value to lower than 1; the acidified system was passed through a diatomite pad having a size of 1 to 2 cm, the pH of an obtained filtrate was regulated to 5 to 6 with 235 g of sodium hydroxide to obtain a crude product aqueous solution. The crude product aqueous solution was passed through a resin column of a strong acid cation exchange resin whose model number is 001X7 to be purified, an obtained crude product was washed with absolute ethyl alcohol and then suction filtration was performed, and an obtained filter cake was dried to obtain an almost white solid product; an obtained filtrate was passed through a resin column of a strong acid cation exchange resin whose model number is 001X7 to be purified to obtain an almost white solid product, 87 g of the two almost white solid products was obtained in total, Nuclear Magnetic Resonance (NMR) internal standard>97%, chiral purity>99% and yield>49%. IH MR(400 MHz, D₂O): δ2.56 (d, 1H), 0.91(m, 1H), 0.49(m, 2H), 0.34(m, 1H), 0.23(m, 1H). MS: (M+H)⁺=116.1.

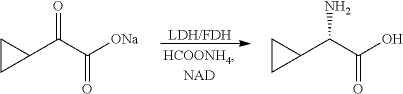

### Embodiment 2

Synthesis of L-cyclobutyl Glycine

Step 1: 15.13 g of magnesium chips, 160 ml of THF, and 2 iodine seeds were added to a 1 L four-neck flask, and then 32 ml of a THF solution having 8 g of bromocyclobutane was added to the flask, the four-neck flask was heated so a bromocyclobutane Grignard reagent in the system was triggered; then the four-neck flask was cooled to 40° C., 288 ml of a THF solution having 72 g of bromocyclobutane was dropwise added, the addition is finished in about 2.5 h, bromocyclobutane was subjected to a reaction according to the following equation, the temperature was increased to about 40 to 50° C., the temperature was preserved for about 1 h to obtain a Grignard reagent and then the Grignard reagent was cooled to room temperature and protected with nitrogen for further use.

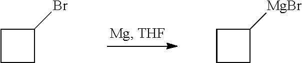

Step 2: 160 ml of a THF solution having 112.6 g of diethyl oxalate was added to a 1 L four-neck flask, the four-neck flask was cooled to a temperature below −50° C. with liquid nitrogen and ethanol, the temperature of the four-neck flask was controlled to below −35° C., 0.593 mol of the prepared Grignard reagent was pressed into the cooled system and a reaction according to the following equation was carried out, the Grignard reagent is added in about 1 h; 1 h later, a sample was subjected to Thin Layer Chromatography (TLC) detection for about 1.5 h until full reaction is achieved; 200 ml of hydrochloric acid having a concentration of 3 mol/L was added to the reaction system to terminate the reaction to obtain a mixed system; then the mixed system was extracted with 160 ml of Methyl Tert-Butyl Ether (MTBE) for three times, and organic phases obtained after the extraction was combined and concentrated to obtain 140 g of a crude product cyclobutyl oxoacetate. IH MR(400 MHz, CD₃Cl): 64.30 (m, 2H), 3.78(m, 1H), 2.20(m, 2H), 2.01(m, 2H), 1.83(m, 2H), 1.33(t, 3H).

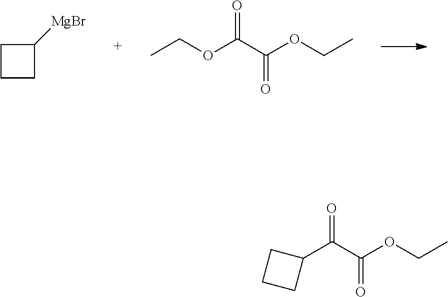

Step 3: 200 ml of a phosphoric acid buffer solution having 100 g of the cyclobutyl oxoacetate and a pH value of 7, and 2 g of bacillus sphaericus were added to a 500 L four-neck flask to form a reaction system, the reaction system was heated to 30 to 40° C., the pH value of the reaction system was maintained to 7 to 8 with ammonia during the reaction process, wherein the specific reaction process is expressed by the following equation; about 50 h later, the TLC detected that the reaction is finished. Suction filtration was performed for the system, an obtained filtrate was extracted with 150 ml of MTBE to obtain an aqueous solution of a keto acid to be used in the reaction of the next step directly. IH MR(400 MHz, D₂O): δ3.66(m, 1H), 2.17 to 1.75 (m, 6H);

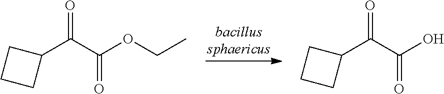

Step 4: 640 g of a cyclobutyl keto acid aqueous solution with a concentration of 6.46% and 17.42 g of sodium hydroxide were added to a 1 L beaker at room temperature, stir and suction filtration were performed to remove a precipitated solid. an obtained filtrate was transferred to a 1 L four-neck flask, 40.65 g of ammonium formate, 400 mL of a crude enzyme mixed solution having a leucine dehydrogenase with a specific enzyme activity of 75 U/ml and a formate dehydrogenase with a specific enzyme activity of 35 U/ml and 2.14 g of β-NAD⁺ were added to the four-neck flask to form a mixed system, the pH value of the mixed system was regulated to 8.0 to 8.5 and the mixed system was heated to 30 to 40° C. so that the mixed system reacts according to the following equation; the mixed system was tracked after reacting for about 44 h, detected that the raw materials finish reaction, the mixed system was cooled to room temperature, 130 mL of concentrated hydrochloric acid was slowly added to the mixed system in a dropwise manner while stirring so that the pH value of the mixed system is smaller than or equal to 1 after the reaction; the acidified system was passed through a diatomite pad having a size of 1 to 2 cm, the pH value of a collected filtrate was regulated to 5 to 6 with sodium hydroxide to obtain a crude product aqueous solution; the crude product aqueous solution was passed through a resin column of a strong acid cation exchange resin whose model number is 001X7, an obtained purified crude product was washed with absolute ethyl alcohol and then suction filtration was performed, and a filter cake was dried to obtain an almost white solid product; a filtrate obtained after the suction filtration was passed through a resin column of a strong acid cation exchange resin whose model number is 001X7 to be purified to obtain an almost white solid product, 24.8 g of the two solid products was obtained in total, NMR internal standard>97%, chiral purity>99% and yield>60%. 1H MR(400 MHz, D₂O): δ3.12 (d, 1H), 2.40(m, 1H), 2.0 to 1.75(m, 6H). MS: (M+H)⁺=130.1.

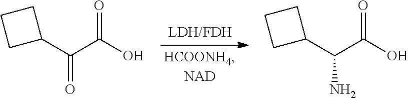 [SMILES: O=C(O)C(=O)C1CCC1 ; N[C@@H](C(=O)O)C1CCC1]

### Embodiment 3

Synthesis of L-cyclopentyl Glycine

Step 1: 300 mL of THF, 97.9 g of Mg, 30 g of bromocyclopentane and 2 iodine seeds were added to a dry 5 L four-neck flask to trigger a reaction, then a mixed solution of 476 g of bromocyclopentane and 2233 mL of THF were dropwise added continually, the temperature was controlled to 60 to 65° C., the mixed solution is dropwise added in about 4 h, reflux was performed for about 2 hours and then cooled to room temperature to obtain a Grignard reagent. 679.9 g of diethyl oxalate and 1176 mL of THF were added to a new dry 5 L four-neck flask, and cooled to below −40° C., and the Grignard reagent was pressed to the reaction system formed in the new four-neck flask, the temperature of the reaction system was controlled to below −40° C., the Grignard reagent was pressed in about 1 h, the temperature was preserved for about 30 min and then the reacted system was heated to room temperature, then the pH value of the system was regulated to 3 to 4 with hydrochloric acid having a concentration of 3 mol/L, and the system was performed liquid separation to obtain a water phase and an organic phase, the water phase was extracted with ethyl acrylate to obtain an organic phase, the two organic phases were combined and then washed with a saturated sodium carbonate solution until the organic phase was neutral, and then the neutral organic phase was washed with saturated salt water and concentration was performed to obtain 413 g of a crude product with a yield of 72%.

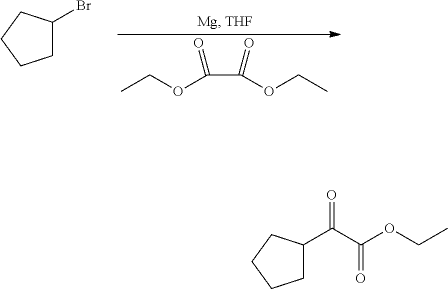

Step 2: 400 g of the crude product and 400 mL of water were added to a 2 L four-neck flask to form a mixed system, then the mixed system was heated to 80° C. and 440 g of a NaOH aqueous solution with a mass concentration of about 50% was dropwise added to the mixed system, the NaOH aqueous solution was added in about 1 h, and then the substances in the four-neck flask reacted according to the following equation, then naturally be cooled to room temperature, the cooled system was performed suction filtration, and an obtained filtrate was extracted with n-heptane to obtain a water phase and an organic phase, the water phase will be used directly in the next step.

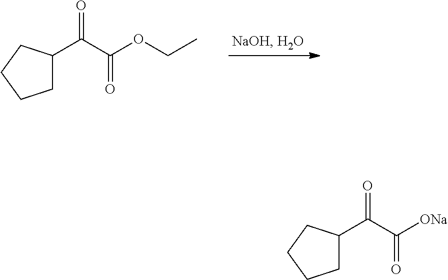

Step 3: the water phase (containing 385.7 g of a sodium salt) obtained in the previous step, 13.74 g of β-NAD⁺, 443.5 g of ammonium formate and 4.36 L of a crude enzyme mixed solution having a leucine dehydrogenase with a specific enzyme activity of 70 U/ml and a formate dehydrogenase with a specific enzyme activity of 35 U/ml were added to a four-neck flask, and stired to dissolve all substances to formed a mixed system, about 10 mL of strong ammonia was added to the mixed system to regulate the pH value of the mixed system to about 8, then heat the mixed system was heated to about 30 to 40° C., after reacting for 3 days, samples were tracked until conversion of the raw materials is finished, totally add 1.2 L of concentrated hydrochloric acid was totally added to the reacted system to regulate the pH value of the system to below 1, the system was passed through diatomite, and the pH of an obtained filtrate was regulated to 5 to 6 with a sodium hydroxide solution having a concentration of 50%, the system was performed suction filtration at a temperature lower than 30° C. to obtain a solid, the solid was dried and then washed with pure water having a volume which is 4 times as large as that of the solid for 2 h while stirring, then an obtained filter cake was performed suction filtration and washed with ethyl alcohol to obtain 134.6 g of a product, internal standard>97%, ee>99% and yield=40%. 1H MR(400 MHz, D₂O): δ2.79 (d, 1H), 1.89(m, 1H), 1.65 to 1.43(m, 6H),2.19(m, 2H). MS: (M+H)⁺=144.1.

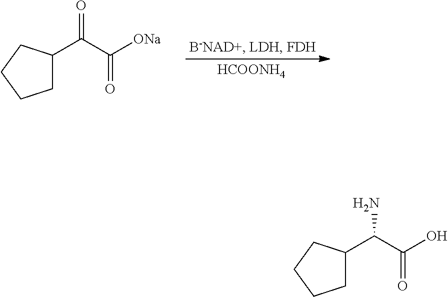

### Embodiment 4

Synthesis of L-cyclohexyl Glycine

Step 1: 3320 g of magnesium chips, 500 mL of THF and 2 g of iodine were added to a 20 L four-neck flask, 1 L of a THF solution having 203 g of bromocyclohexane at 20° C. was dropwise added to the 20 L four-neck flask, and stired until a system was triggered; after the triggering, a mixed solution of 1.947 kg of bromocyclohexane and 10 L of THF was dropwise added by batches, the temperature was controlled at 55 to 60° C. after the addition, the temperature was preserved at 60 to 65° C. for 2 h, and then stopped heating, and an obtained Grignard reagent was protected and stored with nitrogen temporarily; 2.07 kg of diethyl oxalate and 2.5 L of THF were added to a dry 5 L reaction bottle to form a mixed system; the mixed system was cooled to a temperature below −40° C. with liquid nitrogen and ethanol, the prepared Grignard reagent was pressed to the mixed system by nitrogen, the temperature was controlled below −20° C. during the dropwise adding process, the Grignard reagent was added in a dropwise manner within about 2 h, the reacted mixed system was heated to −10±5° C. and the temperature was preserved for 1 h; the reacted mixed system was cooled to −20° C. and about 1.08 L of concentrated hydrochloric acid was dropwise added to regulate the pH value of the system to 4 to 5; then 1.5 L of water and 0.5 L of ethyl acetate were added to the system, the system was stired and then separated to obtain a water phase and an organic phase, the water phase was extracted twice with 800 mL of ethyl acetate respectively, the organic phase was washed twice with 1 L of a saturated sodium bicarbonate solution and then washed twice with 1.5 L of saturated salt water, and the organic phase was concentrated to obtain 4.41 kg of a yellow liquid, i.e. cyclohexyloxo ethyl acetate.

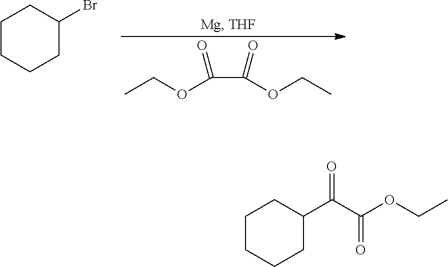

Step 2: 4.12 kg of cyclohexyloxo ethyl acetate and 1.79 kg of methanol were added to a 20 L reaction bottle, and stired until they were fully dissolved, 5.454 kg of water was added to the reaction bottle continually, the temperature of the system was slightly increased; 2.56 kg of a prepared sodium hydroxide solution having a mass concentration of 50% was dropwise added, the temperature was increased, and the sodium hydroxide solution was added in a dropwise manner within about 40 min, reaction was acted for 3 h while preserving the temperature at 50 to 60° C. Track was performed until the raw materials were fully reacted, the system was cooled to 42° C., suction filtration was performed to remove an insoluble solid and a filter cake and a filtrate were obtained, the filter cake was washed with 700 mL of water, and then a water wash liquid was collected; impurities were extracted from the filtrate twice with 7 L of MTBE and 6 L of MTBE respectively, a MTBE phase was discarded, and the pH value of a water phase was regulated with 1.6 L of concentrated hydrochloric acid so that the pH value of the system is 1 to 2; then extraction was performed twice with 5 L of dichloromethane and 2 L of dichloromethane respectively until there is no product in the water phase, obtained dichloromethane phases were combined, and the obtained dichloromethane phase was dried overnight with 0.5 kg of anhydrous sodium sulfate, suction filtration was performed, and then an obtained filter cake was soaked with 1 L of dichloromethane, an obtained filter cake was discarded and an obtained filtrate was concentrated, the concentrated liquid was dissolved with methanol and a sodium methylate solution was dropwise added to it, the liquid was stired for 30 min in an ice water bath and then suction filtration was performed to obtain 1.69 kg of a white solid (i.e. sodium 2-cyclohexyl-2-oxyacetate); 1H MR(500 MHz, D₂O): δ2.88 (m, 1H), 1.88(m, 2H), 1.74 to 1.04(m, 8H);

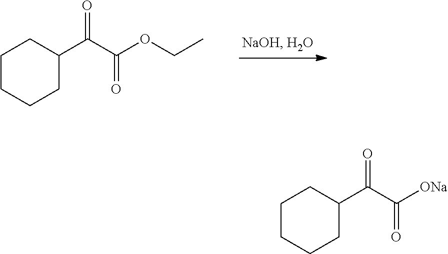

Step 3: at room temperature, 996 g of HCOONH₄ and 2.58 kg of purified water were added to a 20 L four-neck flask,and stired to dissolve, and then 7.556 kg of a crude enzyme mixed solution having a leucine dehydrogenase with a specific enzyme activity of 60 U/ml and a formate dehydrogenase with a specific enzyme activity of 40 U/ml, 701 g of sodium 2-cyclohexyl-2-oxyacetate and 24.8 g of β-NAD⁺ were added to the system. After they were fully dissolved, 47 g of strong ammonia was added to the system to regulate the pH value of the system to 8.0 to 8.5, the temperature was increased to 30 to 40° C. and reacted overnight, after reacting completely, 1.05 L of concentrated hydrochloric acid was dropwise added to the system to terminate the reaction, then the system was passed through a diatomite pad having a size of 1 to 2 cm to obtain a filter cake and a filtrate, the filter cake was washed twice with 0.7 L of purified water to obtain a water wash liquid, and then the water wash liquid and the filtrate were transferred to a 20 L four-neck flask to form a mixed solution, the pH value of the mixed solution was regulated to 5 to 6 with 1.1 L of a sodium hydroxide solution having a mass concentration of 50%, suction filtration was performed to the mixed solution to obtain a filter cake and a filtrate, the filter cake was wash twice with 2 L of purified water, and then the filter cake was washed with water having a volume which is 5 times as large as that of the filter cake for three times while stirring, and the filter cake was dried to obtain 512.8 g of a product having a yield of 82.9%, NM internal standard>98%, and chiral purity>99%. 1H MR(400 MHz, D₂O): δ3.54 (d, 1H), 1.61(m, 1H), 1.38 to 1.25(m, 5H), 0.91 to 0.69 (m, 5H).

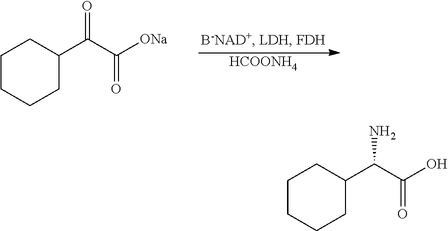

### Embodiment 5

Synthesis of L-cycloheptyl Glycine

Step 1: 12.9 g of magnesium chips, 180 ml of THF, and 1 iodine seed and 36 ml of a THF solution having 9 g of cycloheptyl bromide were added to a 1 L four-neck flask to form a reaction system, the reaction system was heated to clarity and triggered, the triggered reaction system released intense heat, the reaction system was cooled to 50° C. and the temperature of the reaction system was controlled to lower than or equal to 50° C., 324 ml of a THF solution having 81 g of cycloheptyl bromide was dropwise added within about 1 h, and the temperature of the system was increased to 45 to 55° C., the temperature was preserved for about 5 h and then cooled to room temperature to obtain a Grignard reagent.

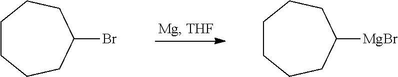

Step 2: 74.6 g of diethyl oxalate and 160 ml of a THF solution were added to a 1 L four-neck flask to form a mixed system, the mixed system was cooled to a temperature lower than or equal to −50° C. with liquid nitrogen and ethanol. The temperature of the mixed system was controlled to a temperature lower than or equal to −30° C., 0.51 mol of the prepared Grignard reagent was pressed into the cooled system, the Grignard reagent was added within about 0.5 h. About 2.5 h later, TLC detection was performed until reacting completely. 40 ml of hydrochloric acid having a concentration of 2 mol/L was added to the system to terminate the reaction and then 40 ml of water was added to perform liquid separation, a water phase obtained through the liquid separation was extracted with 250 ml of ethyl acetate to obtain an organic phase, the organic phase obtained through the liquid separation and the organic phase obtained through the extraction were combined and then washed with 50 ml of water for three times and with 50 ml of saturated salt water in turn, and the organic phases was concentrated to obtain 98.0 g of a cycloheptyloxo ethyl acetate crude product.

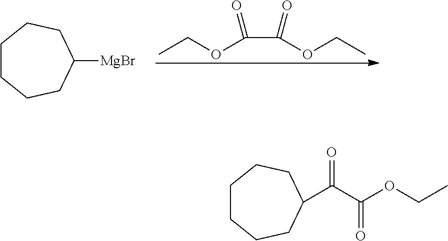

Step 3: 98.0 g of the cycloheptyloxo ethyl acetate crude product, 196 ml of methanol and 368 ml of a sodium hydroxide solution having a concentration of 2 mol/L were added to a 1 L four-neck flask to form a reaction system, the reaction system released heat until the temperature is 50° C., the temperature was preserved at 40 to 50° C., about 5 h later, TLC detection was performed until reacting completely. The system was concentrated until there were substantially no fractions, 320 ml of water was added, and then extraction was performed twice with 100 ml of petroleum ether respectively, the pH value of an obtained water phase was regulated to about 2 with hydrochloric acid having a concentration of 6 mol/L, and extraction was performed twice with 120 ml of MTBE, and four times with 120 ml of ethyl acetate respectively to obtain organic phases, the organic phases was combined and washed three times with 120 ml of water, and once with 50 ml of saturated salt water, the organic phase was concentrated to obtain a solid, nitrogen was blew to dry the solid to obtain 29 g of a keto acid. 1H MR(400 MHz, CD₃Cl): δ8.95 (s, 1H), 3.36(m, 1H), 1.93(m, 2H), 1.74(m, 2H), 1.63 to 1.51(m, 8H).

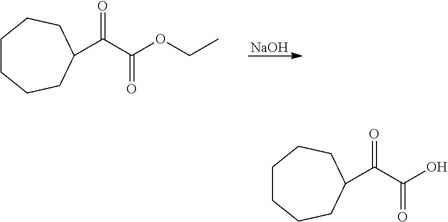

Step 4: 20.9 g of the crude product keto acid was solved in 60 ml of methanol, 27.43 g of a methanol solution of sodium methylate having a mass concentration of 29% was dropwise added in a ice water bath. After dropwise adding the methanol solution, stir was performed for 10 min in an ice water bath, suction filtration was performed, an obtained filter cake was washed with 50 L of EA to obtain a white solid, the white solid was dried and 18.68 g of a keto acid sodium salt was weighed. 90 mL of pure water was added to 18 g of the keto acid sodium salt, and then filtered to remove insoluble matters, an obtained filtrate was transferred to a 500 mL four-neck flask. 11.8 g of ammonium formate, 180 mL of a crude enzyme mixed solution having a leucine dehydrogenase with a specific enzyme activity of 100 U/ml and a formate dehydrogenase with a specific enzyme activity of 50 U/ml, and 0.62 g of β-NAD⁺ were added to the four-neck flask form a reaction system, then the pH value of the reaction system was regulated to 8.0 to 8.5 with strong ammonia, then the reaction system was heated to 30 to 40° C. and reacted overnight, 400 mL of concentrated hydrochloric acid was slowly added to the four-neck flask in a dropwise manner to terminate the reaction, the system was passed through diatomite, the pH of an obtained filtrate was regulated to 5 to 6 with solid sodium hydroxide in an ice water bath, and stired for 30 min and then suction filtration was performed to obtain a white solid; the white solid was washed with 10 mL of water and then dried to obtain 3.9 g of L-cycloheptyl glycine, internal standard>97% and chiral purity>99%. 1H MR(400 MHz, D₂O): δ3.08 (d, 1H), 1.77(m, 1H), 1.63 to 1.26(m, 12H).

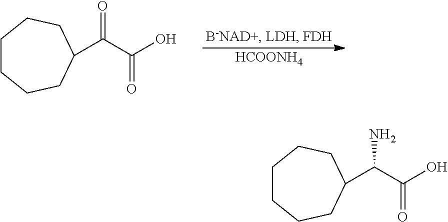 [SMILES: O=C(O)C(=O)C1CCCCCC1 ; "B-NAD+, LDH, FDH" ; "HCOONH4" ; N[C@H](C(=O)O)C1CCCCCC1]

### Embodiment 6

Synthesis of L-cyclohexyl Alanine

Step 1: 11 g of magnesium chips, 30 ml of THF, and 4 g of bromocyclohexane were added to a 500 ml four-neck flask to form a mixed system, the mixed system was heated to 30 to 40° C. and then 2 iodine seeds were added to the mixed system to trigger the system, then a mixture of the remaining 61 g of bromocyclohexane and 230 mL of THF was dropwise added. micro-refluxing was performed during the dropwise addition which is finished in 1 h, the temperature of the system was preserved at 60 to 70° C. for 2 h and then cooled to room temperature for further use and a Grignard reagent was obtained.

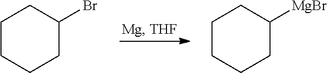

Step 2: The prepared Grignard reagent was pressed into a dry and clean 20 L reaction bottle, the temperature of the Grignard reagent was cooled to −20° C. and controled at −10±5° C. then 1.506 g of dimethylformamide was dropwise added to the reaction bottle in about 2 h, the temperature of the system was preserved at a temperature below 0° C. for 2 h. 7 L of HCl having a concentration of 4 mol/L was dropwise added to the reaction bottle to terminate the reaction to obtain a reaction product. Suction filtration was performed for the reaction product, an obtained filtrate was standed to enable liquid separation, an obtained water phase was extracted twice with 2.8 L of EA, extracted organic phases were combined and washed with 3 L of saturated salt water, and concentration was performed to obtain 1.608 kg of an oily liquid having a purity of 96% and a yield of 83%. 1H MR(400 MHz, CDCl₃): δ9.49 (s, 1H), 2.13(m, 1H), 1.74(m,2H), 1.66 to 1.51(m, 4H), 1.22 to 1.10 (m, 4H).

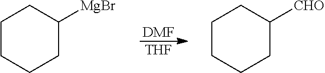

Step 3: 272 g of the oily liquid and 1632 mL of methanol was added to a 5 L four-neck flask, the temperature was cooled to below 0° C. and 92 g of a sodium borohydride was added by batches, the temperature was controlled below 30° C. The adding sodium borohydride was finished in about 2 h, the system reacted at 20 to 30° C. while preserving the temperature. HCl having a concentration of 4 mol/L was dropwise added to the system so that the pH value of the system was 6 to 7, then preliminary concentration was performed for the system and toluene was added to the preliminarily-concentrated system to further concentrate the system to obtain 325.9 g of a crude product, reduced pressure distillation was performed to obtain 214.7 g of a colorless liquid, p=99%, with a yield of 82%. IH MR(400 MHz, CDCl₃): δ3.36 (d, 2H), 2.48(s, 1H), 1.69 to 1.60(m,4H), 1.4(m, 1H), 1.24 to 1.07 (m, 4H),0.88 (m, 2H).

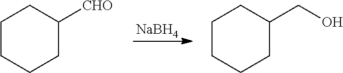 [SMILES: O=CC1CCCCC1 ; "NaBH4" ; OCC1CCCCC1]

Step 4: 1.4 kg of the colorless liquid, 8.4 L of toluene and 969.8 g of pyridine were added to a 20 L four-neck flask, the system was cooled to 0 to 10° C., then a mixture of 1.66 kg of phosphorus tribromide and 7 L of toluene was dropwise added, the temperature was controlled below 5° C. The dropwise addition was finished in about 1 h, the temperature was increased to room temperature and reacted for 10 h. Then the temperature was cooled to below 20° C., about 2.5 L of a sodium hydroxide solution having a concentration of 5% was dropwise added, then 1.85 kg of solid sodium hydroxide was added to form a mixture. Liquid separation was performed for the mixture, an obtained water phase was extracted twice with 4 L of toluene, organic phases obtained after the extraction were combined and washed with saturated salt water, the washed organic phases was dried with anhydrous sodium sulfate and then concentrated to obtain 1.73 kg of a crude product, reduced pressure distillation was performed to obtain 722.8 g of a product having a purity of 97.5% and a yield of 33.3%. 1H MR(400 MHz, CDCl₃): δ3.82 (m, 1H), 1.79 to 1.53(m,6H), 1.13 to 0.90 (m, 6H).

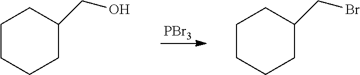

Step 5: 85.4 g of magnesium chips, 500 mL of THF and 30 g of the product obtained in Step 4 were added to a 5 L four-neck flask, the system was heated to trigger, then a mixed liquid of 570 g of the product obtained in Step 4 and 3.4 L of THF was dropwise added, after the dropwise addition, the temperature was preserved at 60±5° C. for 4 h, and cooled to room temperature for further use to obtain a Grignard reagent. 594.2 g of diethyl oxalate and 1.2 L of THE were added to another 10 L reaction bottle, stired and cooled to −40 to −50° C., then the prepared Grignard reagent was dropwise added in about 2 h, reaction was performed at −10 to −20° C. for about 1.5 h to obtain a product, then 600 mL of HCl having a concentration of 6 mol/L was dropwise added to terminate the reaction and the pH value of the product was regulated to 2 to 3, then 500 mL of water was added, a water phase was extracted for three times with 1.2 L of MTBE to obtain organic phases, the organic phases were combined, washed with saturated salt water, and concentrated to obtain 935 g of a brown liquid used directly in the reaction of the next step.

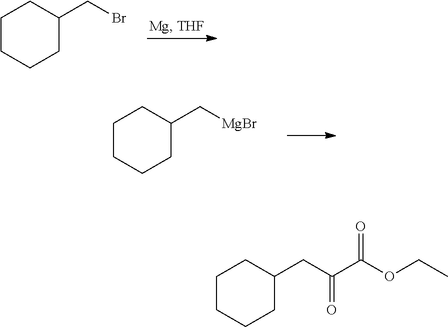

Step 6: 935 g of the brown liquid obtained in Step 5 and 839 ml of pure water were added to a 3 L four-neck flask, then a mixed solution of 203.1 g of NaOH and 203.1 g water was dropwise added to the four-neck flask, the temperature was controlled below 40° C. The dropwise addition of the mixed solution was finished in about 2 h, reaction of the system in the four-neck flask was performed for about 5 h and then suction filtration was performed, the pH value of an obtained filtrate was regulated with HCl to 1 to 2, then the filtrate was extracted for three times with 1 L of EA respectively, the organic phases obtained after the extraction was concentrated and dropwise added to a sodium methylate solution to prepare a sodium salt for further use, 198 g of the sodium salt was obtained in total with a yield of 30%. IH MR(400 MHz, CDCl₃): δ2.63 (d, 2H), 1.8(m, IH), 1.63 to 1.56 (m, 5H), 1.24 to 1.06(m,3H), 0.91 (m, 2H).

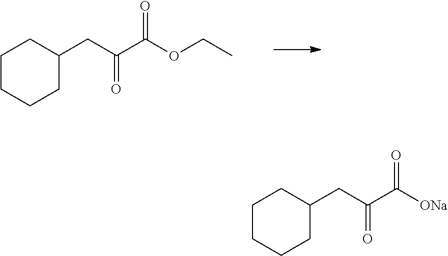

Step 7: at room temperature, 74 g of the sodium salt obtained in the previous step and 740 mL of purified water were added to a 5 L four-neck flask, an ultrasonic treatment was performed at 20° C. for 5 min and then filtered, 48.5 g of ammonium formate, 740 mL of a crude enzyme mixed solution having a leucine dehydrogenase with a specific enzyme activity of 50 U/ml and a formate dehydrogenase with a specific enzyme activity of 30 U/ml, and 2.55 g of β-NAD⁺ were added to an obtained filtrate to form a reaction system, the pH value of the reaction system was detected to be 6 to 7, the pH value of the reaction system was regulated to 8.0 to 8.5 with 20 mL of ammonia having a mass concentration of 5%, the temperature of the reaction system was increased to 30° C. to 40° C., reaction was performed for 3 days, then the system was cooled to room temperature, 220 mL of concentrated hydrochloric acid was slowly added in a dropwise manner to the flask to regulate the pH value of the system to 1 to 2, the system was passed through a diatomite having a size of 1 to 2 cm, the pH value of an obtained filtrate was regulated to 5 to 6 with 101.5 g of sodium hydroxide, the filtrate was put on the upper layer of a refrigerator to crystallize, suction filtration was performed to obtain a crude product. The crude product was washed with purified water having a volume which is 5 times as large as that of the crude product for three times, and washed with absolute ethyl alcohol having a volume which is three times as large as that of the crude product twice, and the crude product was dried to obtain 23.7 g of a product internal standard>98% and yield >99%. IH MR(400 MHz, D₂O): δ3.15 (m, 1H), 1.61 to 1.53(m, 5H), 1.36 (m, 1H), 1.23(m, 2H), 1.08 to 1.00 (m, 3H), 0.79 (m, 2H).

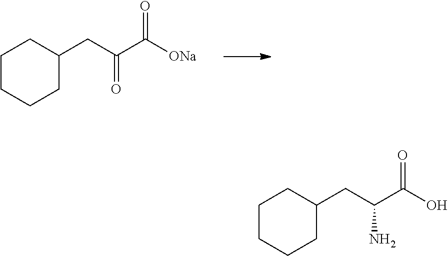

### Embodiment 7

L-4′-pyranyl Glycine

Step 1: 6.2 g of 4-carboxypyran and 30 mL of THF were added to a 250 mL of four-neck flask and stired until they were dissolved. The temperature was cooled to −10 to 0° C. in an ice salt bath, 81.04 g of an IPMgCl/THF solution having a concentration of 12.8% was dropwise added while controlling the temperature, dropwise addition the solution was finished within about 1.5 h, then 5.31 g of diisopropylamine was rapidly added at a time, then the temperature was increased and refluxed for 2 h; the system was cooled to −30 to −20° C., T was controlled to T<−5° C., 7.66 g of diethyl oxalate was dropwise added in about 15 min, then the temperature was naturally increased to room temperature and reaction was performed for 45 min; the system was cooled to 0 to 5° C., 2.45 g of EtOH was dropwise added, stired for 30 min after the dropwise addition, then 13 mL of concentrated hydrochloric acid was dropwise added, the temperature was preserved at 50° C. for 50 min, then cooled to room temperature, 20 mL of water and 30 mL of MTBE were added, stir was performed for 10 min and then liquid separation was performed, an obtained water phase was extracted twice with 30 mL of MTBE respectively, organic phases obtained after the extraction were combined, and the combined organic phase was washed with 20 mL of 1M HCl hydrochloric acid and 20 mL of saturated salt water in turn, the washed organic phase was dried with MgSO₄ and then concentrated to obtain 7.34 g of a liquid; 1H MR(400 MHz, CD₃Cl): δ4.30 (m, 2H), 3.97(t, 2H), 3.44(t, 2H), 3.28(m, 1H), 1.80(t, 2H), 1.72(m, 2H), 1.36 (t, 3H).

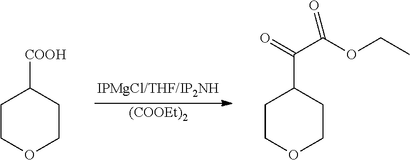

Step 2: 10 mL of water, 1.26 g of KOH and 1.16 g of K₂HPO₄were added to a 50 mL three-neck flask, the temperature was controlled at T<20° C. a mixed liquid of 2.5 g of the keto acid ester liquid obtained in the previous step and 5 mL of MeOH was dropwise added, after the dropwise addition, the temperature was controlled at T<20° C. and preserved for 1 h. 10 Ml of MTBE was added to the system and stired for 10 min, and then perform liquid separation was performed, a water phase obtained through the liquid separation was extracted with 10 mL of MTBE, the pH value of the extracted water phase was regulated to 3 to 4 with 1.4 mL of 6N HCl, then the water phase was extracted twice with 20 mL of EA respectively, organic phases obtained after the extraction was combined and concentrated to obtain 0.72 g of a yellow oily substance, i.e. a keto acid crude product; 1H MR(400 MHz, CD₃Cl): δ4.08 (t, 2H), 3.54(t, 2H), 3.50(m, 1H), 1.87(m, 2H), 1.72(m, 2H).

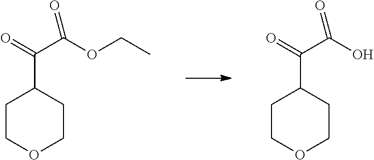

Step 3: 0.4 g of the keto acid crude product was added to 5 mL of Iso-Propyl Alcohol (IPA), 0.58 g of a sodium methylate solution having a concentration of 28% was dropwise added and stired for 5 min in an ice bath and then suction filtration was performed to obtain 0.5 g of an almost white solid; the almost white solid was added to a 50 mL Erlenmeyer flask, 2.5 mL of pure water and 0.26 g of ammonium formate were added, the pH was regulated to 8.0 to 8.5 with strong ammonia, then 4 mL of a crude enzyme mixed solution having a leucine dehydrogenase with a specific enzyme activity of 60 U/ml and a formate dehydrogenase with a specific enzyme activity of 20 U/ml, and 18.4 g of β-NAD⁺ were added, reaction was performed overnight at a constant temperature of 30° C. The pH value of the system was regulated to pH<1 with 1 mL of concentrated hydrochloric acid, the system was passed through diatomite and suction filtration was performed, the pH value of an obtained filtrate was regulated to 5 to 6 with solid NaOH in an ice bath, and the filtrate with the pH value of 5 to 6 was passed through a strong acid cation exchange resin to purify a product to obtain an almost white solid, ee>99%. IH MR(400 MHz, D₂O): δ3.99 (t, 2H), 3.44(t, 2H), 3.00 (d, 1H), 1.75(m, 1H), 1.57 (m, 2H), 1.33 (m, 2H).

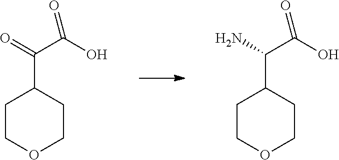 [SMILES: O=C(O)C(=O)C1CCOCC1 ; N[C@H](C(=O)O)C1CCOCC1]

### COMPARISON EXAMPLE 1

A process for reducing a benzene ring with L-phenylalanine to obtain a product, which is disclosed in a patent W02005/14526 A1, was applied.

L-phenylalanine was added to 200 mL of water, 200 mL of isopropanol, 12.2 mL of hydrochloric acid having a concentration of 37% and 2 g of Pt/Rh(4:1) carbon having a concentration of 50% were added, hydrogen was introduced to the system, the pressure of the system was maintained at 8 to 10 bar, reaction was performed at 50° C. to 60° C. for 6 to 8 h. Then the system was filterred and washed with 50 mL of water, a filtrate was concentrated and then the pH value of the filtrate was regulated to 5 to 6 with sodium hydroxide having a concentration of 50%. Then the temperature was cooled to 0 to 10° C., suction filtration was performed to obtain a solid and wash the solid with 20 mL of water, then vacuum drying was performed at 50 to 70° C.,

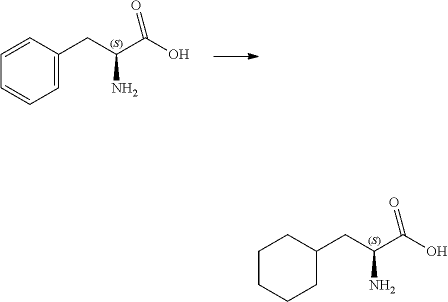

### COMPARISON EXAMPLE 2

A method disclosed in an American patent U.S. Pat. No. 6,191,306 was used for synthesizing cyclopropyl glycine.

Step 1: 175 g of cyclopropyl formaldehyde was dissolved in 1.75 g of methanol, then 322 mL of S-α-methylbenzylamine was added. Reflux was performed for 1.5 h and then the temperature was cooled to 30° C., then 162.8 g of edit potassium cyanide was added, reaction was performed and the temperature was increased to 32° C., stired overnight, then 580 mL of water was added. The pH value of the system was regulated to 10 with 40 mL of concentrated hydrochloric acid, and 1.8 L of water was added, a water phase was separated and extracted with EA for 3 times, 0.7 L of EA is used each time. Organic phases were combined and then dried with anhydrous magnesium sulfate, then the organic phase was filtered, and a filtrate was concentrated to obtain a yellow oily substance with a yield of 98%. Product (S,S): (R,R)=3.2:1;

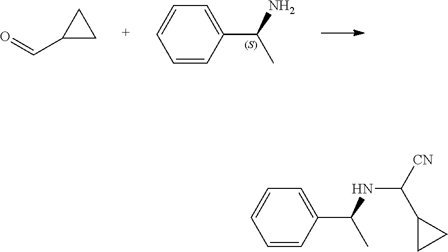

Step 2: 10 g of the product of Step 1 was dissolved in 65 mL of concentrated hydrochloric acid, the temperature increased to 94° C. and was preserved for 17 h, then cooled to below room temperature, 175 mL of a 4N potassium hydroxide solution was dropwise added until the pH value of the system is 8 to 9. Then the system was stirred in an ice bath for 45 min and filtered to obtain a solid, the solid was washed with 50 mL of ice water, then washed with 100 mL of ice methanol for 10 min. then the solid was filter to obtain a white solid, the white solid was washed twice with 50 mL of methanol respectively, then filtering and vacuum drying were performed to obtain a product having a yield of 55% and a chiral purity higher than 98%;

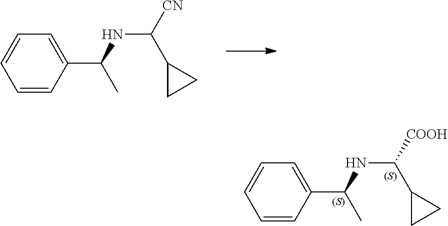

Step 3: 16.8 g of (S)-phenylethyl-(S)-cyclopropyl glycine obtained in the previous step, 200 mL of THE, 100 mL of water, and 4.76 g of Pd/C having a concentration of 10% were mixed, then 17 mL of formic acid was added, then stir was performed overnight. After reacting, filtering was performed to remove a catalyst to obtain a filtrate, the filtrate was concentrated, and then methanol was added, then concentration was performed over again, operate repeatedly for several times, and vacuum drying was performed to obtain 4.75 g of a solid product.

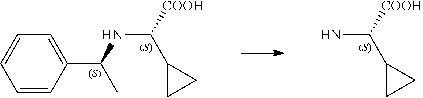

Chiral purities of L-cyclic alkyl amino acids of the 1^{st} embodiment to the 7^{th} embodiment and the 1^{st} Comparison example to the 2^{nd} Comparison example were obtained through NMR internal standards and recorded in Table 1.

TABLE 1

| | | | | | | | | Compar- | Compar- |
|---|---|---|---|---|---|---|---|---|---|
| | | | | | | | | ison | ison |
| | Embodi- | Embodi- | Embodi- | Embodi- | Embodi- | Embodi- | Embodi- | exam- | exam- |
| | ment 1 | ment 2 | ment 3 | ment 4 | ment 5 | ment 6 | ment 7 | ple 1 | ple 2 |
| Chiral | >99 | >99 | >99 | >99 | >99 | >99 | >99 | >98 | >99 |
| purity | | | | | | | | | |

It can be learned from the data in Table 1 that the chiral purities of the L-cyclic alkyl amino acids obtained by a preparation method of the present disclosure in the 1^{st} embodiment to the 7^{th} embodiment are higher than 99%. In addition, an L-cyclic alkyl amino acid can be obtained by a synthesis method of the present disclosure in two steps by taking a keto acid as a substrate, thus the synthesis route is shorter than that of the 2^{nd} comparison file. In addition, a heavy metal catalyst is applied in synthesis processes of the 1^{st} comparison example and the 2^{nd} comparison example, which results in high synthesis cost. The synthesis method of the 2^{nd} comparison example causes waste of part of the raw materials and results in high synthesis cost.

The above are only preferred embodiments of the present disclosure and should not be used for limiting the present disclosure. For those skilled in the art, the present disclosure may have various modifications and changes. Any modifications, equivalent replacements, improvements and the like within the spirit and principle of the present disclosure shall fall within the scope of protection of the present disclosure.

1-19. (canceled)

20. A synthesis method for L-cyclic alkyl amino acid, wherein the synthesis method comprises:
Step A: preparing a cyclic alkyl keto acid or a cyclic alkyl keto acid salt having Structural Formula (I) or Structural Formula (II);
Step B: mixing the cyclic alkyl keto acid or the cyclic alkyl keto acid salt with an ammonium formate, a leucine dehydrogenase, a formate dehydrogenase and a coenzyme NAD⁺, and carrying out a reductive amination reaction to generate the L-cyclic alkyl amino acid, wherein
the Structural Formula (I) is 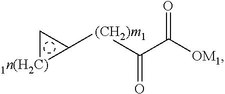
where n₁≧1, m₁≧0 and the M₁ is H or a monovalent cation;
the Structural Formula (II) is 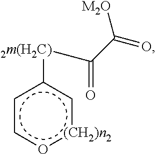
where n₂≧0, m_{2≧0,} the M₂ is H or a monovalent cation; an amino acid sequence of the leucine dehydrogenase is SEQ ID No. 1.

21. The synthesis method according to claim 20, wherein a gene sequence coding the leucine dehydrogenase is SEQ ID No. 2.

22. The synthesis method according to claim 21, wherein an expression process of the leucine dehydrogenase comprises:
inserting a DNA fragment containing the gene sequence SEQ ID No. 2 into a vector to obtain a gene recombinant plasmid;
transferring the gene recombinant plasmid to a host strain and culturing the host strain on a culture medium, and inducing production of the leucine dehydrogenase by an inducer;
breaking the host strain with ultrasonic waves, and then carrying out centrifugal separation to obtain a crude enzyme mixed solution which contains the leucine dehydrogenase and the formate dehydrogenase, wherein the specific enzyme activity of the leucine dehydrogenase is 50 U/ml to 100 U/ml and the specific enzyme activity of the formate dehydrogenase is 20 U/ml to 50 U/ml.

23. The synthesis method according to claim 21, wherein the Step B comprises:
adding the cyclic alkyl keto acid or the cyclic alkyl keto acid salt and ammonium formate to water, mixing and then regulating the pH value to 8.0 to 8.5 to obtain a mixed solution, adding the crude enzyme mixed solution and the coenzyme NAD⁺ to the mixed solution and performing reaction at 30 to 40° C. to generate the L-cyclic alkyl amino acid.

24. The synthesis method according to claim 22, wherein the Step B comprises:
adding the cyclic alkyl keto acid or the cyclic alkyl keto acid salt and ammonium formate to water, mixing and then regulating the pH value to 8.0 to 8.5 to obtain a mixed solution, adding the crude enzyme mixed solution and the coenzyme NAD⁺ to the mixed solution and performing reaction at 30 to 40° C. to generate the L-cyclic alkyl amino acid.

25. The synthesis method according to claim 23, wherein
2 ml to 12 ml of the crude enzyme mixed solution is added to each mole of the cyclic alkyl keto acid;
0.005 mole to 0.1 mole of the coenzyme NAD⁺ is added to each mole of the cyclic alkyl keto acid and 1.5 to 5 moles of the ammonium formate is added to each mole of the cyclic alkyl keto acid.

26. The synthesis method according to claim 24, wherein
2 ml to 12 ml of the crude enzyme mixed solution is added to each mole of the cyclic alkyl keto acid;
0.005 mole to 0.1 mole of the coenzyme NAD is added to each mole of the cyclic alkyl keto acid and 1.5 to 5 moles of the ammonium formate is added to each mole of the cyclic alkyl keto acid.

27. The synthesis method according to claim 20, wherein after the Step B, the method further comprises:
adding concentrated hydrochloric acid to a system after the reaction to regulate the pH value of the system to be smaller than or equal to 1, passing the system with the pH value of smaller than or equal to 1 through diatomite to obtain a filtrate;
regulating the pH value of the filtrate to 5.0 to 7.0, then passing the filtrate with the pH value of 5.0 to 7.0 through a strong acid cation exchange resin to obtain a crude product;
concentrating the crude product, adding an alcoholic solvent to wash the concentrated crude product and drying the washed crude product to obtain a purified L-cyclic alkyl amino acid.

28. The synthesis method according to claim 20, wherein a method for preparing the cyclic alkyl keto acid having the Structural Formula (I) comprises the following steps:
preparing a Grignard reagent of a cyclic alkyl halogen compound having Structural Formula (III);
subjecting the Grignard reagent and a diethyl oxalate to a substitution reaction to obtain an intermediate product;
subjecting the intermediate product to a hydrolysis reaction to obtain the cyclic alkyl keto acid,
wherein the Structural Formula (III) is 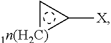
where n₁≧1 and the X is a halogen.

29. The synthesis method according to claim 28, wherein the n₁ in the Structural Formula (III) is 1 or 2, and the intermediate product is subjected to the hydrolysis reaction under the action of a biological enzyme.

30. The synthesis method according to claim 28, wherein the n₁ in the Structural Formula (III) is larger than or equal to 3, and the intermediate product is subjected to the hydrolysis reaction under the action of a sodium hydroxide or a potassium hydroxide.

31. The synthesis method according to claim 20, wherein a method for preparing the cyclic alkyl keto acid salt having the Structural Formula (I) comprises:
preparing a Grignard reagent of the cyclic alkyl halogen compound having Structural Formula (III);
subjecting the Grignard reagent and a diethyl oxalate to a substitution reaction to obtain an intermediate product;
subjecting the intermediate product to a hydrolysis reaction to obtain a cyclic alkyl keto acid; enabling the cyclic alkyl keto acid to react with CH₃OM₁′ or M₁′OH to obtain the cyclic alkyl keto acid salt,
wherein the Structural Formula (III) is 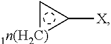
where n₁≧1, the X is a halogen and the M1′ is a monovalent cation.

32. The synthesis method according to claim 31, wherein in the Structural Formula (III), 3≦n₁≦5, and the intermediate product is subjected to the hydrolysis reaction under the action of a sodium hydroxide or a potassium hydroxide.

33. The synthesis method according to claim 20, wherein the cyclic alkyl keto acid salt is a cyclopropyl keto acid salt and a method for preparing the cyclopropyl keto acid salt comprises the following step: oxidizing cyclopropyl methyl ketone in an alkaline condition to obtain the cyclopropyl keto acid salt.

34. The synthesis method according to claim 33, wherein KMnO₄ is applied as an oxidant in the oxidation process and the oxidant is reduced until it disappears after the oxidation reaction.

35. The synthesis method according to claim 20, wherein the method for preparing the cyclic alkyl keto acid salt having the Structural Formula (I) comprises the following steps:
subjecting cyclic alkyl formaldehyde to a reduction reaction to obtain cyclic alkyl methyl alcohol;
subjecting the cyclic alkyl methyl alcohol to a halogenation reaction to obtain a second cyclic alkyl methyl halogen compound having Structural Formula (IV);
preparing a Grignard reagent of the second cyclic alkyl methyl halogen compound;
subjecting the Grignard reagent and a diethyl oxalate to a substitution reaction to obtain an intermediate product;
subjecting the intermediate product to a hydrolysis reaction to obtain a cyclic alkyl keto acid;
enabling the cyclic alkyl keto acid to react with CH₃OM₁′ or M₁′OH to obtain the cyclic alkyl keto acid salt,
wherein the Structural Formula (IV) is 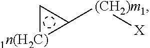
where n₁≧1, m₁=1, the X is a halogen, and the M₁′ is a monovalent cation.

36. The synthesis method according to claim 35, wherein the method for preparing the cyclic alkyl keto acid salt having the Structural Formula (I) further comprises a process for preparing the cyclic alkyl formaldehyde, and the preparing process comprises:
preparing a Grignard reagent of a cyclic alkyl halogen compound having Structural Formula (III);
enabling the Grignard reagent to have a Bouveault aldehyde synthesis reaction to obtain the cyclic alkyl formaldehyde,
wherein the Structural Formula (III) is 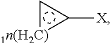
where n₁≧1, the X is a halogen.

37. The synthesis method according to claim 20, wherein a method for preparing the cyclic alkyl keto acid having the Structural Formula (II) comprises the following steps:
subjecting carboxylic acid having Structural Formula (V) to a Grignard reagent addition reaction to obtain an intermediate product;
subjecting the intermediate product to a hydrolysis reaction in an alkaline condition to obtain the cyclic alkyl keto acid,
wherein the Structural Formula (V) is 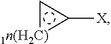
where n₂≧0 and m₂>0.

38. The synthesis method according to claim 37, wherein n₂=1 and m₂=0.

39. A pharmaceutical composition wherein it comprises an effective dose of an L-cyclic alkyl amino acid and a pharmaceutical vector, the L-cyclic alkyl amino acid is prepared according to the synthesis method according to claim 1.
